(19) United States
(12) Patent Application Publication
(10) Pub. No.: US 2021/0221792 A1
(43) Pub. Date: Jul. 22, 2021

(54) CONVERGENT SYNTHESIS OF POZIOTINIB DERIVATIVE

(71) Applicant: Hanmi Pharm Co., Ltd., Gyeonggi-do (KR)

(72) Inventors: Bang Keuk Chan, Gyeonggi-do (KR); Jung Jae Hyuk, Gyeonggi-do (KR); Moon Young Ho, Gyeonggi-do (KR)

(21) Appl. No.: 17/149,901

(22) Filed: Jan. 15, 2021

Related U.S. Application Data
(60) Provisional application No. 62/961,884, filed on Jan. 16, 2020.

(51) Int. Cl. C07D 401/14; C07D 401/12

(52) U.S. Cl. CPC C07D 401/14; C07D 401/12

(57) ABSTRACT

A method of for the synthesis of a compound of Formula I is disclosed herein. The method includes reacting a compound of formula (II) with a compound of formula (III) in an inert polar aprotic solvent system in the presence of a base.

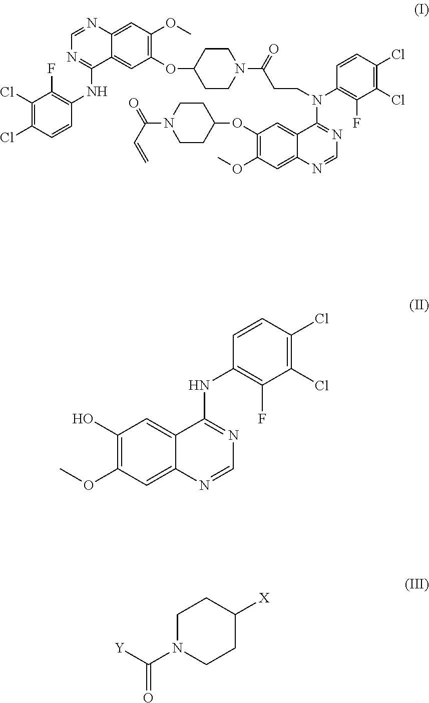

### TECHNICAL FIELD

Disclosed herein is a novel process for the preparation of a poziotinib derivative.

### BACKGROUND

Poziotinib, having a chemical name of 1-(4-(4-(3,4-dichloro-2-fluorophenylamino)-7-methoxyquinazolin-6-yloxy)piperidin-1-yl)prop-2-en-1-one, is known to exhibit anti-proliferative activities such as anti-cancer activities. The CAS Registry No. of the compound is 1092364-38-9.

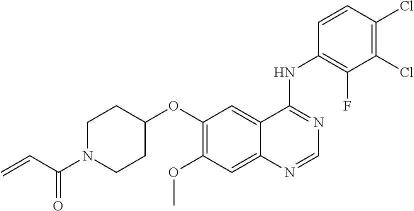 [SMILES: C=CC(=O)N1CCC(Oc2cc3c(Nc4ccc(Cl)c(Cl)c4F)ncnc3cc2OC)CC1]

While the synthesis of the above compound has been disclosed in international patent application No. PCT/KR2014/000752, the preparation of its analogs or derivatives depends on the specific structure of the target compound and needs to be explored on a case-by-case basis.

### SUMMARY

This document discloses a novel synthesis of a quinazoline compound (Formula I) which bears two substituted quinazoline components. By employing suitable polar aprotic solvent in the final displacement step, the target compound can be generated as detailed herein.

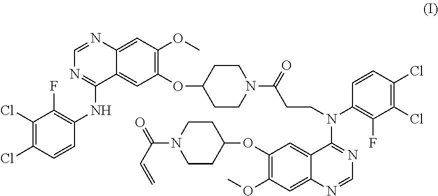 [SMILES: C=CC(=O)N1CCC(Oc2cc3c(N(CCC(=O)N4CCC(Oc5cc6c(Nc7ccc(Cl)c(Cl)c7F)ncnc6cc5OC)CC4)c4ccc(Cl)c(Cl)c4F)ncnc3cc2OC)CC1]

The preparation of the compound of Formula I generally involves reacting a compound of formula (II) with a compound of formula (III) in an inert polar aprotic solvent system in the presence of a base. The group X is tosyloxy (OTs), mesyloxy (OMs), trifluoromethane sulfonate, fluorosulfonate or halogen; and the group Y is ethenyl or halogenoethyl.

Suitable inert polar aprotic solvent system includes acetonitrile, acetone, dichloromethane, chloroform, carbon tetrachloride, 1,4-dioxane, ethyl acetate, tetrahydrofuran, and any combination thereof. In some embodiments, the inert polar aprotic solvent system includes at least one selected from acetonitrile, acetone, dichloromethane, chloroform, carbon tetrachloride, 1,4-dioxane, ethyl acetate, and tetrahydrofuran. In some embodiments, the inert polar aprotic solvent system further comprises a solvent selected from the group consisting of N,N-dimethylformamide, N,N-dimethyl acetamide, N-methylpyrrolidin-2-one, and dimethyl sulfoxide.

In some embodiments, the base is an alkali metal carbonate selected from the group consisting of sodium bicarbonate, potassium carbonate, cesium carbonate and a mixture thereof. In some embodiments, the base is used in an amount of 1 to 5 mole equivalents based on 1 mole equivalent of the compound of formula (II).

In some embodiments, the compound of formula (II) is prepared by (i) subjecting a compound of formula (VII) to a reaction with a halogenating agent in the presence of an organic base to produce the compound of formula (VI), which is then subjected to a reaction with a compound of formula (VIII) to obtain the compound of formula (V); and (ii) subjecting the compound of formula (V) to a reaction with an ammonia solution in a polar protic solvent:

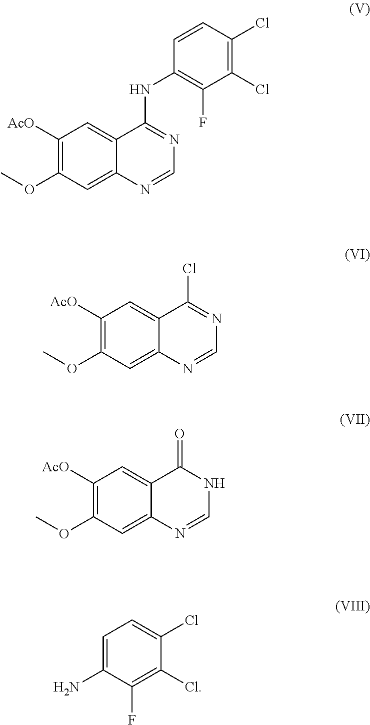 [SMILES: COc1cc2ncnc(Nc3ccc(Cl)c(Cl)c3F)c2cc1OC(C)=O ; COc1cc2ncnc(Cl)c2cc1OC(C)=O ; COc1cc2nc[nH]c(=O)c2cc1OC(C)=O ; Nc1ccc(Cl)c(Cl)c1F]

In some embodiments, the reaction step (i) employs an organic base selected from the group consisting of diisopropylamine, triethylamine, diisopropylethylamine, diethylamine, pyridine, 4-dimethylpyridine, morpholine and a mixture thereof. In some embodiments, the reaction step (i) involves a halogenating agent selected from the group consisting of thionyl chloride, phosphorus oxychloride and a mixture thereof.

In some embodiments, the polar protic solvent is selected from the group consisting of methanol, ethanol, propanol and a mixture thereof.

In some embodiments, the compound of formula (III) is prepared by allowing the compound of formula (IX) or its salt to react with the compound of formula (X) in the presence of a base or an amide coupling agent, wherein X and Y are the same as defined in claim 1; and Z is halogen or hydroxyl. In some embodiments, the salt of the compound of formula (IX) is a hydrochloride or hydrobromide salt.

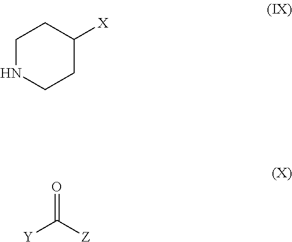

In some embodiments, the reaction between the compound of formula (IX) or its salt and the compound of formula (X) is conducted in an organic solvent or a mixture of an organic solvent and water; and the organic solvent is tetrahydrofuran, ethyl acetate, acetone, 1,4-dioxane, acetonitrile, dichloromethane, carbon tetrachloride, chloroform, N,N-dimethyl formamide or dimethylsulfoxide.

In some embodiments, the base used in the reaction between the compound of formula (IX) or its salt and the compound of formula (X) is selected from the group consisting of sodium carbonate, sodium bicarbonate, calcium carbonate, potassium carbonate, sodium hydroxide, potassium hydroxide, cesium carbonate, diisopropylamine, triethylamine, diisopropylethylamine, diethylamine, and a mixture thereof.

In some embodiments, the amide coupling agent is selected from the group 1-ethyl-3-(3-dimethylaminopropyl)carbodiimide, hydroxybenzotriazole, O-(7-azabenzotriazol-1-yl)-N,N,N′,N′-tetramethyluronium hexafluorophosphate, N,N′-dicyclohexylcarboimide, 1-hydroxy-7-azabenzotriazole, N—N′-diisopropylcarboimide, (benzotriazol-1-yloxy)tris(dimethylamino)phosphonium hexafluorophosphate and a mixture thereof.

In some embodiments, the method also produces compound I′.

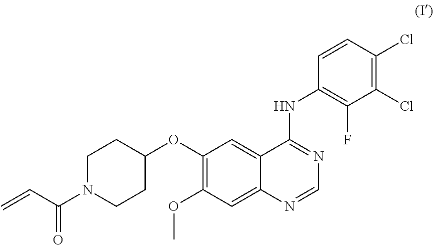 [SMILES: C=CC(=O)N1CCC(Oc2cc3c(Nc4ccc(Cl)c(Cl)c4F)ncnc3cc2OC)CC1]

In some embodiments, the methods includes acidifying the reaction mixture after the reaction and converting the reaction product to a salt form. In some embodiments, the salt form is obtained by reacting the compound (I) with an acid selected from the group consisting of HCl, HBr, and MsOH.

### DETAILED DESCRIPTION

This patent document discloses the novel synthesis of Compound I. The compound includes key structural components of poziotinib, which is known to exhibit anticancer activities. Through a beta elimination process, compound I splits into two poziotinib molecules.

This preparation of compound I is shown below. Compound II and III react with each other in the presence of a polar aprotic solvent to produce compound I.

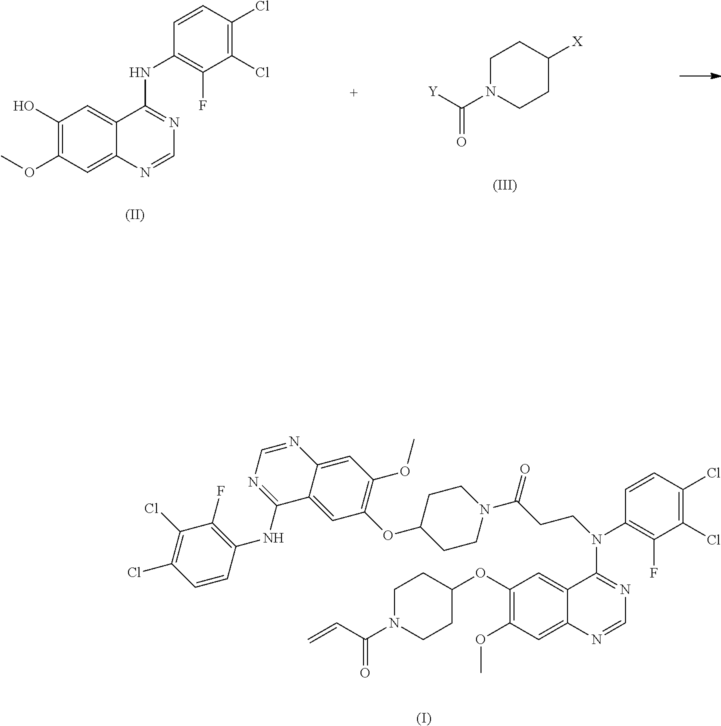

Without being limited to any particular theory, the reaction is postulated to go through the formation of compound I′, which then form compound I via inter-molecular Michael addition.

Suitable inert solvent for the reaction solvent system generally includes at least acetonitrile, acetone, dichloromethane, chloroform, carbon tetrachloride, 1,4-dioxane, ethyl acetate, tetrahydrofuran, or any combination thereof. Any of these solvents may also be used in combination with one or more of N,N-dimethylformamide, N,N-dimethylacetamide, N-methylpyrrolidin-2-one, dimethyl sulfoxide. In some embodiments, from about 0.1% to about 99%, from about 1% to about 90%, from about 1% to about 50%, from about 1% to about 90%, from about 1% to about 50%, from about 1% to about 20%, or from about 1% to about 10% of the solvent system is acetonitrile, acetone, dichloromethane, chloroform, carbon tetrachloride, 1,4-dioxane, ethyl acetate, tetrahydrofuran, or any combination thereof. In some embodiments, the solvent system contains at least 5%, at least 10%, at least 20%, at least 50%, at least 80%, or at least 95% of N,N-dimethylformamide, N,N-dimethylacetamide, N-methylpyrrolidin-2-one, dimethyl sulfoxide, or any combination thereof.

The reaction takes place in the presence of an inorganic base or an organic base. In some embodiments, the base used in the above reaction is alkali metal carbonates such as sodium bicarbonate, potassium carbonate, cesium carbonate and a mixture thereof. In some embodiments, the base is used in an amount of 1 to 5 mole equivalents based on 1 mole equivalent of the compound of formula (II).

The temperature of the reaction ranges from 20° C. to 150° C. In some embodiments, the temperature ranges from about 60° C. to about 100° C., from about 70° C. to about 90° C., or from about 70° C. to about 80° C.

The compound of formula (II), which is used as a starting material in the present invention, can be prepared by the following steps:
(i) subjecting a compound of formula (VII) to a reaction with a halogenating agent in the presence of an organic base to produce the compound of formula (VI), which is then subjected to a reaction with a compound of formula (VIII) to obtain the compound of formula (V), i.e., 4-(3,4-di chloro-2-fluorophenylamino)-7-methoxyquinazolin-6-yl acetate; and
(ii) subjecting the compound of formula (V) to a reaction with an ammonia solution in a polar protic solvent.

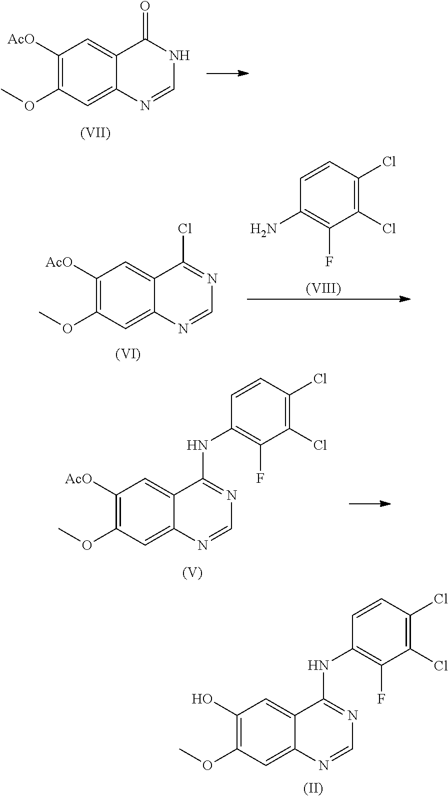

Non-limiting examples of the organic base used in Step (i) above include diisopropylamine, triethylamine, diisopropylethylamine, diethylamine, pyridine, 4-dimethylpyridine, morpholine and a mixture thereof. Examples of the halogenating agent include thionyl chloride, phosphorus oxychloride and a mixture thereof. The above reaction may be conducted at 50° C. to 150° C., preferably 60° C. to 90° C., more preferably at about 75° C. In this step, the compound of formula (VI) is prepared in the form of a solution containing it in an organic solvent, rather than an isolated form. Subsequently, the compound of formula (VI) contained in the organic solvent is allowed to react with the compound of formula (VIII) to obtain the compound of formula (V), i.e., 4-(3,4-dichloro-2-fluorophenylamino)-7-methoxyquinazolin-6-yl acetate.

The compound of formula (VII), which is used as a starting material of the above reaction, can be prepared by the method disclosed in Korean Patent No. 1013319.

In the subsequent step (ii), the compound of formula (V) prepared in the previous step (i) is allowed to react with an ammonia solution or ammonia gas in a polar protic solvent (e.g., methanol, ethanol, propanol and a mixture thereof) at a temperature of 0° C. to 40° C., preferably 10° C. to 30° C., more preferably at about 25° C., to obtain 4-(3,4-dichloro-2-fluorophenylamino)-7-methoxyquinazolin-6-ol of formula (II).

The compound of formula (III), which is used as a synthetic intermediate, can be prepared by allowing the compound of formula (IX) or its salt to react with the compound of formula (X) in the presence of a base or an amide coupling agent as shown below

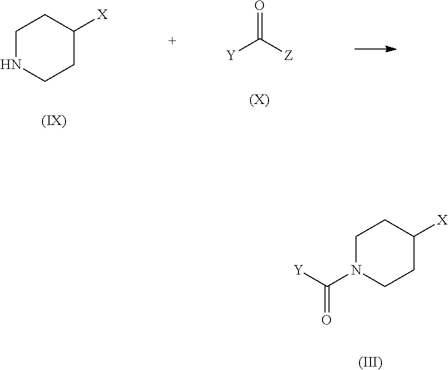

wherein X and Y are the same as defined above; and Z is halogen or hydroxyl.

The above reaction can be conducted in an organic solvent such as tetrahydrofuran, ethyl acetate, acetone, 1,4-dioxane, acetonitrile, dichloromethane, carbon tetrachloride, chloroform, N,N-dimethyl formamide or dimethylsulfoxide, or in a mixture of an organic solvent and water.

Non-limiting examples of the base include an inorganic base such as sodium carbonate, sodium bicarbonate, calcium carbonate, potassium carbonate, sodium hydroxide, potassium hydroxide and cesium carbonate, an organic base such as diisopropylamine, triethylamine, diisopropylethylamine and diethylamine, and a mixture thereof. Examples of the amide coupling agent include 1-ethyl-3-(3-dimethylaminopropyl)carbodiimide, hydroxybenzotriazole, O-(7-azabenzotriazol-1-yl)-N,N,N′,N′-tetramethyluronium hexafluorophosphate, N,N′-dicyclohexylcarboimide, 1-hydroxy-7-azabenzotriazole, N—N′-diisopropylcarboimide, (benzotriazol-1-yloxy)tris(dimethylamino)phosphonium hexafluorophosphate and a mixture thereof. The base or amide coupling agent may be used in an amount of 3 to 5 mole equivalents based on 1 mole equivalent of the compound of formula (IX) or a salt thereof.

The salt of the compound of formula (IX) above is preferably a hydrochloride salt (2HCl salt) or a hydrobromide salt (2HBr salt). The above reaction may be conducted at a temperature of −30° C. to 30° C., preferably about 0° C. to room temperature, by stirring for a suitable period of time.

Besides the compound of Formula I, the synthesis approach disclosed herein also produces the compound of Formula I′. The ratio between the two compounds may vary depending on factors such as the specific solvent and the base.

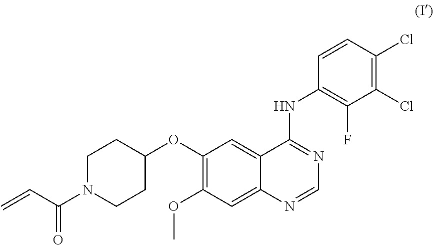 [SMILES: C=CC(=O)N1CCC(Oc2cc3c(Nc4ccc(Cl)c(Cl)c4F)ncnc3cc2OC)CC1]

In some embodiments, the synthesis further includes converting the product mixture of the compounds of Formula I and Formula I′ to a salt form. For instance, the product mixture can react with hydrochloric acid in an organic solvent (e.g., methanol, ethanol, propanol, isopropanol, butanol, ethyl acetate, acetone, tetrahydrofuran, acetonitrile, 1,4-dioxane and a mixture thereof) at a temperature of 0° C. to 60° C., preferably 10° C. to 40° C., more preferably at about 25° C. Other acidic agents that can be used for the preparation of the salt form include HBr, and MsOH. The amount of the acid may be for example more than 1 mole equivalent, more than 2 mole equivalents, more than 3 mole equivalents, more than 5 mole equivalents, more than 10 mole equivalents, or more than 20 mole equivalents of the amount of the compound of Formula I and/or Formula I′.

The specific conditions such as reaction temperature, the amount of a reagent, and other relevant reaction factors in each of the above described reactions may vary. One skilled in art would be able to practive the synthesis methods of this patent document without undue experiments in view of the instant disclosure and the general knowledge in the field of organic chemistry. For instance, the preparation of certain intermediates of this patent document can be prepared in view of the examples of PCT/KR2014/000752, the entire disclosure of which is hereby incorporated by reference.

While the forgoing text may reference or exemplify specific embodiments of a reaction step or a method of preparing an intermediate, it is not intended to limit the scope of the method to such particular reference or examples. Various modifications may be made by those skilled in the art, in view of practical and economic considerations, such as the amount of the individual intermediates or reagent in the reaction and the length of time of conducting the reaction.

As used herein, the articles “a” and “an” refer to “one or more” or “at least one,” unless otherwise indicated. That is, reference to any element or component of an embodiment by the indefinite article “a” or “an” does not exclude the possibility that more than one element or component is present.

As used herein, the term “about” generally refers to plus or minus 10% of the indicated number. For example, “about 10%” may indicate a range of 9% to 11%, and “about 20” may mean from 18 to 22. Other meanings of “about” may be apparent from the context, such as rounding off, so, for example “about 1” may also mean from 0.5 to 1.4. As used herein, the term “and/or” includes any and all combinations of one or more of the associated listed items. Expressions such as “at least one of,” when preceding a list of elements, modify the entire list of elements and do not modify the individual elements of the list. When referring to a dosing protocol, the term “day”, “per day” and the like, refer to a time within one calendar day which begins at midnight and ends at the following midnight.

It will be appreciated by persons skilled in the art that invention described herein are not limited to what has been particularly shown and described. Rather, the scope of the invention is defined by the claims which follow. It should further be understood that the above description is only representative of illustrative examples of embodiments. The description has not attempted to exhaustively enumerate all possible variations. The alternate embodiments may not have been presented for a specific component of the drug combination, or a step of the method, and may result from a different combination of described constituents, or that other un-described alternate embodiments may be available for a combination or method, is not to be considered a disclaimer of those alternate embodiments. It will be appreciated that many of those un-described embodiments are within the literal scope of the following claims, and others are equivalent.

What is claimed is:

1. A method for preparing the compound of formula (I), which comprises the step of allowing the compound of formula (II) to react with the compound of formula (III) in an inert polar aprotic solvent system in the presence of a base: 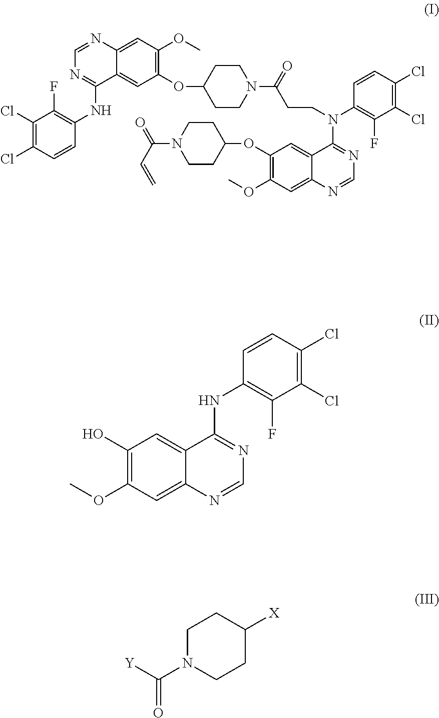
wherein X is tosyloxy (OTs), mesyloxy (OMs), trifluoromethane sulfonate, fluorosulfonate or halogen; and Y is ethenyl or halogenoethyl, wherein the inert polar aprotic solvent system comprises at least one solvent selected from the group consisting of acetonitrile, acetone, dichloromethane, chloroform, carbon tetrachloride, 1,4-dioxane, ethyl acetate, and tetrahydrofuran.

2. The method of claim 1, wherein the inert polar aprotic solvent system further comprises a solvent selected from the group consisting of N,N-dimethylformamide, N,N-dimethylacetamide, N-methylpyrrolidin-2-one, and dimethyl sulfoxide.

3. The method of claim 1, wherein the base is an alkali metal carbonate selected from the group consisting of sodium bicarbonate, potassium carbonate, cesium carbonate and a mixture thereof.

4. The method of claim 1, wherein the base is used in an amount of 1 to 5 mole equivalents based on 1 mole equivalent of the compound of formula (II).

5. The method of claim 1, wherein the compound of formula (II) is prepared by (i) subjecting a compound of formula (VII) to a reaction with a halogenating agent in the presence of an organic base to produce the compound of formula (VI), which is then subjected to a reaction with a compound of formula (VIII) to obtain the compound of formula (V); and (ii) subjecting the compound of formula (V) to a reaction with an ammonia solution in a polar protic solvent:

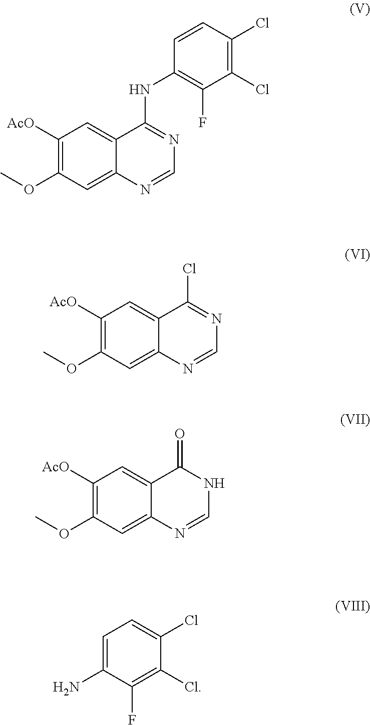 [SMILES: COc1cc2ncnc(Nc3ccc(Cl)c(Cl)c3F)c2cc1OC(C)=O ; COc1cc2ncnc(Cl)c2cc1OC(C)=O ; COc1cc2nc[nH]c(=O)c2cc1OC(C)=O ; Nc1ccc(Cl)c(Cl)c1F]

6. The method of claim 5, wherein the organic base is selected from the group consisting of diisopropylamine, triethylamine, diisopropylethylamine, diethylamine, pyridine, 4-dimethylpyridine, morpholine and a mixture thereof.

7. The method of claim 5, wherein the halogenating agent is selected from the group consisting of thionyl chloride, phosphorus oxychloride and a mixture thereof.

8. The method of claim 5, wherein the polar protic solvent is selected from the group consisting of methanol, ethanol, propanol and a mixture thereof.

9. The method of claim 1, wherein the compound of formula (III) is prepared by allowing the compound of formula (IX) or its salt to react with the compound of formula (X) in the presence of a base or an amide coupling agent, 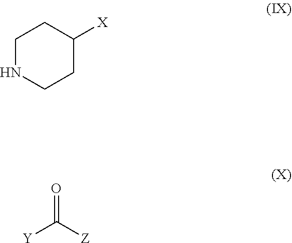
wherein X and Y are the same as defined in claim 1; and Z is halogen or hydroxyl.

10. The method of claim 9, wherein the reaction between the compound of formula (IX) or its salt and the compound of formula (X) is conducted in an organic solvent or a mixture of an organic solvent and water; and the organic solvent is tetrahydrofuran, ethyl acetate, acetone, 1,4-dioxane, acetonitrile, dichloromethane, carbon tetrachloride, chloroform, N,N-dimethyl formamide or dimethylsulfoxide.

11. The method of claim 9, wherein the base used in the reaction between the compound of formula (IX) or its salt and the compound of formula (X) is selected from the group consisting of sodium carbonate, sodium bicarbonate, calcium carbonate, potassium carbonate, sodium hydroxide, potassium hydroxide, cesium carbonate, diisopropylamine, triethylamine, diisopropylethylamine, diethylamine, and a mixture thereof.

12. The method of claim 9, wherein the amide coupling agent is selected from the group 1-ethyl-3-(3-dimethylaminopropyl)carbodiimide, hydroxybenzotriazole, O-(7-azabenzotriazol-1-yl)-N,N,N′,N′-tetramethyluronium hexafluorophosphate, N,N′-dicyclohexylcarboimide, 1-hydroxy-7-azabenzotriazole, N—N′-diisopropylcarboimide, (benzotriazol-1-yloxy)tris(dimethylamino)phosphonium hexafluorophosphate and a mixture thereof.

13. The method of claim 9, wherein the salt of the compound of formula (IX) is a hydrochloride or hydrobromide salt.

14. The method of claim 1, wherein the method also produces compound I′.

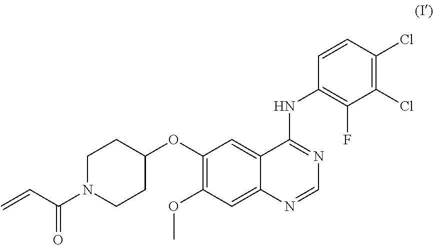 [SMILES: C=CC(=O)N1CCC(Oc2cc3c(Nc4ccc(Cl)c(Cl)c4F)ncnc3cc2OC)CC1]

15. The method of claim 1, further comprising converting compound (I) to a salt form.

16. The method of claim 15, wherein the salt form is obtained by reacting the compound (I) with an acid selected from the group consisting of HCl, HBr, and MsOH.
